(19) United States
(12) Patent Application Publication
(10) Pub. No.: US 2012/0195868 A1
(43) Pub. Date: Aug. 2, 2012

(54) NUTRITIONAL COMPOSITIONS COMPRISING FIBER AND PROBIOTICS

(75) Inventors: Gaby Lathan, Westhofen (DE); James Malcom Grosjean, Ontario (CA); Norman Alan Greenberg, New Hope, MN (US)

(73) Assignee: NESTEC S.A., Vevey (CH)

(21) Appl. No.: 13/387,419

(22) PCT Filed: Jul. 26, 2010
(86) PCT No.: PCT/US10/43189

§ 371 Date: Mar. 27, 2012

(30) Foreign Application Priority Data
Jul. 27, 2009 (US) 61228693

(51) Int. Cl. A61K 35/74; A61P 3/00; A61P 1/12; A61P 17/00; A61P 37/08; A23L 1/305; A61P 3/10; A61P 39/06; A61P 3/02; A23L 1/308; A23L 1/302; A23L 1/304; A61P 1/00; A61P 11/06

(52) U.S. Cl. 424/93.45; 426/61

(57) ABSTRACT

Nutritional compositions including fiber blends having a stable amount of probiotics and methods of making the nutritional compositions are provided. In a general embodiment, the present disclosure provides a nutritional composition including a fiber blend having agglomerated fiber particulates and a probiotic. The fiber blend can have a water activity of less than about 0.15. The nutritional composition can be in an administerable form such as pharmaceutical formulations, nutritional formulations, dietary supplements, functional foods and beverage products.

### BACKGROUND

The present disclosure generally relates to health and nutrition. More specifically, the present disclosure relates to nutritional compositions including fiber blends having a stable amount of probiotics and methods of making the fiber blends.

Probiotics micro-organisms (hereinafter “probiotics”) are micro-organisms that beneficially affect a host by improving its intestinal microbial balance. In general, it is believed that these micro-organisms inhibit or influence the growth and/or metabolism of pathogenic bacteria in the intestinal tract. The probiotics may also activate the immune function of the host. For this reason, there have been many different approaches to include probiotics into food products.

The incorporation of probiotics into food products, however, entails a number of difficulties. One difficulty is having or maintaining an adequate number of viable colony forming units (“cfu's”) per day in the food product. If the concentration of the viable probiotics in the food product does not exceed a certain threshold value, the beneficial effect of the probiotics is not provided. Another concern is the viability of the probiotics in the stomach and the intestine. The probiotics must be sufficiently resistant to the acid environment in the stomach and to the bile acids in order to successfully colonize the intestine. Furthermore, the food product including the probiotics must be palatable to the consumer.

### SUMMARY

Nutritional compositions including fiber blends having a stable amount of probiotics and methods of making the fiber blends are provided. In a general embodiment, the present disclosure provides a nutritional composition including a fiber blend having a mixture of agglomerated fiber particulates and one or more probiotics. The fiber blend has a water activity (“Aw”) of less than about 0.2, In another embodiment, the Aw is less than about 0.15. In a further embodiment, the Aw is in the range is of 0.08-0.2.

The agglomerated fiber particulates can include an average diameter ranging between about 1.2 mm to about 5 mm. The fiber blends in embodiments of the present disclosure can provide for the delivery of the fibers in the same package as the probiotic supplying the probiotic benefits,

In an embodiment, the agglomerated fiber particulates include a fiber such as fructooligosaccharides, inulin, galactooligosaccharides, partially hydrolyzed guar gum, galactomannans, acacia gum, pectins, arabinogalactans, beta-glucans, xanthan gum or a combination thereof. In alternative embodiments, the probiotic can be from a genus such as Aerococcus, Aspergillus, Bacillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, or a combination thereof. In an embodiment, the fiber blend further includes a metabolite generated by the probiotic during a fermentation process.

The nutritional composition can be in an administerable form such as pharmaceutical formulations, nutritional formulations, dietary supplements, functional foods and beverage products. The nutritional composition can further include one or more ingredients such as vitamins, minerals, proteins, bioactives or a combination thereof.

In another embodiment, the present disclosure provides a method for making a nutrition composition. The method includes providing a fiber, agglomerating the fiber to produce agglomerated fiber particulates having a water activity of less that 0.15, and mixing a probiotic with the agglomerated fiber particulates to produce a fiber blend. In an embodiment, the fiber is premixed with a powdered form of the fiber before being mixed with the agglomerated fiber particulates.

In an embodiment, the method can further include storing the fiber blend in a nitrogen atmosphere. The fiber blend can readily be incorporated into pharmaceutical or nutritional formulations (e.g. nutraceuticals), dietary supplements, functional foods and beverage products.

An advantage of the present disclosure is to provide an improved fiber blend having probiotics.

Another advantage of the present disclosure is to provide a method of making a fiber blend having a stable amount of probiotics.

Yet another advantage of the present disclosure is to provide a single product including fiber and a probiotic having an increased shelf-life.

Still another advantage of the present disclosure is to provide a fiber blend having a probiotic that is viable for over 12 months.

Additional features and advantages are described herein, and will be apparent from the following Detailed Description and the figures.

### BRIEF DESCRIPTION OF THE FIGURES

FIG. 1 is a schematic flowchart of a process for making the fiber blend including the agglomerated fiber particulates and the probiotic in an embodiment.

FIG. 2 is a graph illustrating the colony forming units per serving of a probiotic over time at 30° C. for two fiber blends.

### DETAILED DESCRIPTION

Nutritional compositions including fiber blends having a stable amount of probiotics and methods of making the fiber blends are provided. The fiber blends can be part of a nutritional composition or the nutritional composition by themselves. In a general embodiment, the fiber blends include one or more fibers and one or more probiotics. The fiber blends can have a reduced water activity. The fiber blends in embodiments of the present disclosure provide increased convenience for a customer by supplying an all-in-one fiber blend that has a probiotic that remains viable for a long period of time.

As used herein, the term “nutritional composition” includes, but is not limited to, complete nutritional compositions, partial or incomplete nutritional compositions, and disease or condition specific nutritional compositions. A complete nutritional composition (i.e. those which contain all the essential macro and micro nutrients) can be used as a sole source of nutrition for the patient. Patients can receive 100% of their nutritional requirements from such complete nutritional composition. A partial or incomplete nutritional composition does not contain all the essential macro and micro nutrients and cannot be used as a sole source of nutrition for the patient. Partial or incomplete nutritional compositions can be used as a nutritional supplement. A disease or condition specific nutritional composition is a composition that delivers nutrients or pharmaceuticals and can be a complete or partial nutritional composition.

In an embodiment, the present disclosure provides a nutritional composition including a fiber blend having agglomerated fiber particulates and one or more probiotics. The fiber blend can include the probiotic in a concentration ranging from about 0.1% to about 2.0% by weight probiotic with the remainder of the fiber blend being the agglomerated fiber particulates. In an alternative embodiment, the fiber blend can include fiber in a powder form (e.g. average particle size less than about 0.1 mm) in combination with the agglomerated fiber particulates.

In a preferred embodiment, the fiber blend includes about 0.4% by weight probiotic and about 99.6% by weight agglomerated fiber particulates. The fiber blend or the nutritional composition can also include the probiotic in an amount ranging from about 1×10⁷ cfu to about 1×10¹¹ cfu per serving such as about 1×10⁹ cfu and 1×10¹⁰ cfu per serving or other designated food or administration unit.

In an embodiment, the agglomerated fiber particulates have an average diameter greater than about 1.2 mm. In another embodiment, the average diameter of the agglomerated fiber particulates ranges from about 1.2 mm to about 5 mm.

In an embodiment, the fiber blend has a water activity of less than about 0.15. The agglomerated fiber particulates can include any suitable water activity of less than about 0.15 such as less than about 0.14, less than about 0.13, less than about 0.12, less than about 0.10, less than about 0.09 less than about 0.08, less than about 0.07, less than about 0.06 and the like.

The fiber in embodiments of the present disclosure can include any suitable probiotics. Non-limiting examples of fibers that can be used include soluble fibers having a low viscosity such that they can be made into a solution for drying and agglomeration. The fibers can include but are not limited to fructooligosaccharides, inulin, galactooligosaccharides, partially hydrolyzed guar gum, galactomannans, acacia gum, pectins, arabinogalactans, beta-glucans, and hydrolysates of longer chain soluble fibers such as xanthan gum. Two or more fibers can also be combined in any suitable ratio.

The fiber can provide the benefits of regularity, both as a means to reduce constipation and diarrhea. This improved regularity can improve the effectiveness of the probiotic. Probiotics can offer the following benefits: improved gastrointestinal (“GI”) health, reduction of infections by blocking other bacteria from adhering to the GI lining and the presence of reuterin produced by Lactobacillus reuteri cells in the GI tract, reduced risk of Helicobacter pylori and gastric ulcers, and the reduction in sick days due to fever and infections.

As used herein, the term “probiotics” includes any micro-organisms having beneficial effects to its host. Therefore, yeasts, moulds and bacteria may be included. EP 0862863, which is incorporated herein by reference, lists some examples for probiotics presently known. A preferred probiotic can be Lactobacillus reuteri Protectis. The probiotics can be in any suitable form such as, for example, a lyophilized or a spray dried form.

According to embodiments of the present disclosure, suitable probiotics include yeasts such as Saccharomyces, Debaromyces, Candida, Pichia and Torulopsis, moulds such as Aspergillus, Rhizopus, Mucor, and Penicillium and Torulopsis and bacteria such as the genera Bifidobacterium, Bacteroides, Clostridium, Fusobacterium, Melissococcus, Propionibacterium, Streptococcus, Enterococcus, Lactococcus, Staphylococcus, Peptostrepococcus, Bacillus, Pediococcus, Micrococcus, Leuconostoc, Weissella, Aerococcus, Oenococcus and Lactobacillus.

Specific examples of suitable probiotics are: Saccharomyces cereviseae (boulardii), Bacillus coagulans, Bacillus licheniformis, Bacillus subtilis, Bifidobacterium animalis, Bifidobacterium bifidum, Bifidobacterium breve, Bifidobacterium infantis, Bifidobacterium longum, Bifidobacterium lactis, Enterococcus faecium, Enterococcus faecalis, Lactobacillus acidophilus, Lactobacillus alimentarius, Lactobacillus casei subsp. casei, Lactobacillus casei Shirota, Lactobacillus curvatus, Lactobacillus delbruckii subsp. lactis, Lactobacillus farciminus, Lactobacillus fermentum, Lactobacillus gasseri, Lactobacillus helveticus, Lactobacillus johnsonii, Lactobacillus paracasei, Lactobacillus reuteri, Lactobacillus rhamnosus (Lactobacillus GG), Lactobacillus sake, Lactobacillus salivarius, Lactobacillus plantarum, Lactococcus lactis, Micrococcus varians, Pediococcus acidilactici, Pediococcus pentosaceus, Pediococcus acidilactici, Pediococcus halophilus, Streptococcus faecalis, Streptococcus thermophilus, Staphylococcus carnosus, and Staphylococcus xylosus.

The agglomerated fiber particulates can be made using any suitable agglomeration process. For example, a powdered fiber can be wetted using a water mist. The wetted powdered fiber then forms larger clusters of fiber and is subjected to a hot air drying that results in the formation of agglomerated fiber particulates having a lower water activity. The agglomerated particles can then be sieved to separate out agglomerated particles having a desired range of average diameters.

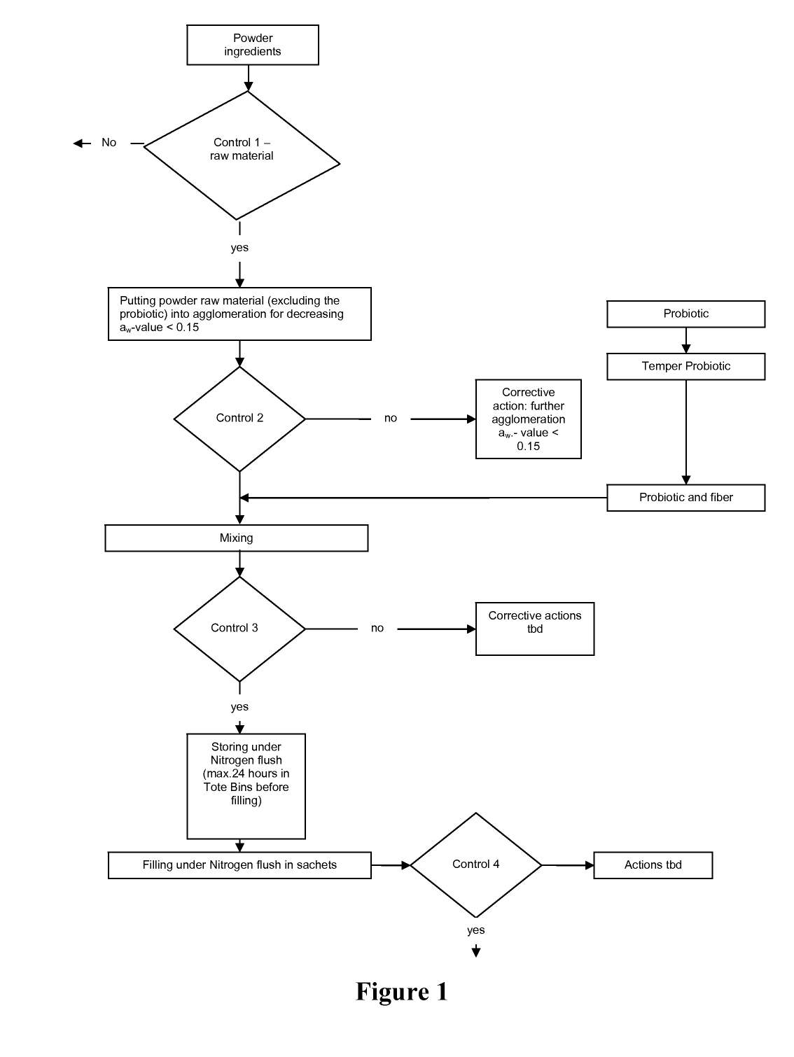

As illustrated in FIG. 1, a method for making the fiber blend in an embodiment includes providing raw fiber materials (e.g. in powder form) that are subjected to quality control testing (e.g. to determine whether safe to use). Once the raw fiber powder passes the quality control testing, it is subjected to an agglomerating process to produce a nutritional composition including a fiber blend including agglomerated have a low water activity.

Detailed steps for agglomerating the raw fiber powder in an embodiment are as follows:
1. Process step: Filling an appropriate container with the raw fiber powder

Air temperature 80° C.; air flow 80%

Target product temperature 40° C.
2. Process step: preheating the raw fiber powder

Air temperature 90° C.; air flow 30%

Target product temperature 43° C.
3. Process step: spraying/misting the raw fiber powder with a suitable fluid (e.g. water) to generate agglomerated fiber particulates

Air temperature 105° C.; air flow 25%

Target product temperature 49/50° C.

Spraying time 11 min
4. Process step: heating the agglomerated fiber particulates (e.g. drying for decreasing the water activity)

Air temperature 105° C.; air flow 20%

Target product temperature 60° C.

heating time (“Drying time”) 10 min
5. Process step: cooling the agglomerated fiber particulates

Room temperature; air flow 20%

Target product temperature 50° C.

Time ˜2 min (depends on real room temperature)

The agglomerated fiber particulates can then be sieved to ensure that the particulates have a specified minimum and maximum average particle size. The agglomerated fiber particulates can then be subject to another quality control to ensure that they have a sufficiently low water activity (e.g. below about 0.15).

The probiotic(s) to be used can be initially premixed with some of the raw fiber powder (e.g. having a particle size less than about 0.1 mm) to separate the probiotics before mixing with the agglomerated fiber particulates. This allows the probiotics to be evenly distributed throughout the agglomerated fiber particulates. The premixed probiotic and raw fiber powder are then mixed with the previously formed agglomerated fiber particulates to form the fiber blend.

By subjecting the raw fiber powder to an agglomerating step as previously described, the fiber blend has been found to have a lower water activity, for example from about 0.05 to about 0.10, than the original raw fiber powder used to make the agglomerated fiber particulates. It has been found that for a temperature of about 25° C., reducing the water activity by an amount of about 0.3 can provide for a 25% increase in shelf-life time (e.g. viability of the probiotic).

The fiber blend can then be packaged and stored under conditions that maintain the viability of the probiotic and water activity of the fiber blend (e.g. nitrogen atmosphere). For example, the packaging can be done with nitrogen flushing to minimize oxygen and moisture from the processing room air. A high quality moisture barrier material can be used in the package. The purpose of the packaging as characterized above is to maintain the preferred water activity values during the shelf life of the fiber blend. The shelf life (e.g. in terms of viability of the probiotic) of the packaged product may be up to 12 months and longer.

The fiber blend can readily be incorporated into any suitable pharmaceutical or nutritional formulations (e.g. nutraceuticals), dietary supplements, functional foods and beverage products. For example, food and beverages for humans as well as pet food may be enriched by the fiber blends. Suitable examples of beverage products include, but are not limited to, water, milk water-based beverages, milk-based beverages, carbonated beverage, non-carbonated beverage, beer, wine, soy milk and fruit-based beverage, like orange juice. Nutritional formulas for each and every purpose may include the fiber blends. A large variety of nutritional formulas exist, for example for sportsmen or athletes, for people with special nutritional needs such as people allergic to certain natural food components or people with gastrointestinal disorders and so forth.

Baked goods, chocolates or other sweet products may include the fiber blends. In addition, all kinds of extruded or cooked or otherwise prepared food products may include the fiber blends. For example, dried products may be used, such as dried pet food or other dried food products like powders, flours, milk or cereal powders or cereal flakes. The fiber blends may be incorporated in all kind of breakfast cereals. In addition, the fiber blends can be added to individual components, ingredients or starting materials of consumable products.

In an embodiment, the fiber blend further includes a metabolite generated by the probiotic during a fermentation process. For example, these metabolites may be released to the medium of fermentation or they may be stored within the microorganism. The metabolites can include part or many of the beneficial effects of a particular probiotic. The nutritional composition can further include one or more ingredients such as vitamins, minerals, proteins, bioactives or a combination thereof.

Non-limiting examples of vitamins include Vitamins A, B-complex (such as B-1, B-2, B-6 and B-12), C, D, E and K, niacin and acid vitamins such as pantothenic acid and folic acid and biotin. Non-limiting examples of minerals include calcium, iron, zinc, magnesium, iodine, copper, phosphorus, manganese, potassium, chromium, molybdenum, selenium, nickel, tin, silicon, vanadium and boron. Non-limiting examples of proteins include peptides, free amino acids, mixtures of amino acids or a combination thereof.

Bioactive compounds or extracts can include those that are known to have health benefits, especially for lowering blood cholesterol level in mammals, such as statins, bile acid sequestrants, nicotinic acid or fibric acids, and/or for treating diabetes, such as sulfonylureas, biguanides, alpha-glucosidase inhibitors, thiazolidinediones, meglitinides or D-phenylalamine and other phytonutrients and antioxidants.

As used herein, non-limiting examples of phytonutrients include those that are flavonoids and allied phenolic and polyphenolic compounds, terpenoids such as carotenoids, and alkaloids; including curcumin, limonin, and quercetin and combinations thereof.

As used herein the term “antioxidant” is preferably understood to include any one or more of various substances (as beta-carotene (a vitamin A precursor), vitamin C, vitamin E, and selenium) that inhibit oxidation or reactions promoted by Reactive Oxygen Species (ROS) and other radical and non-radical species. Additionally, antioxidants are molecules capable of slowing or preventing the oxidation of other molecules. Non-limiting examples of antioxidants include carotenoids, coenzyme Q10 (“CoQ10”), flavonoids, glutathione Goji (Wolfberry), hesperidine, Lactowolfberry, lignan, lutein, lycopene, polyphenols, selenium, vitamin A, vitamin B1, vitamin B6, vitamin B12, vitamin C, vitamin D, vitamin E, and combinations thereof.

The nutritional compositions of the present disclosure can optionally include conventional food additives, such as any of emulsifies, stabilizes, sweeteners, flavorings, coloring agents, preservatives, chelating agents, osmotic agents, buffers or agents for pH adjustment, acidulants, thickeners, texturizers, and so on.

Depending on particularities and preferences, the nutritional compositions including the fiber blends may be exposed to ambient or elevated temperatures, in a way that no substantial loss of probiotic cfu is taken into account. It is also possible to freeze the nutritional composition, depending on its nature or purpose of the final food product. Cold storage without freezing is another method. Of course, other further treatments or processing of the nutritional compositions may occur, depending on the end-product or the purpose of the nutritional compositions. An example would be the aeration of the final nutritional composition with an inert gas or gas mixture like N₂ or N₂/CO₂.

The nutritional compositions of the present disclosure may be administered under the supervision of a medical specialist, or may be self-administered. The nutritional compositions of the present disclosure may be administered daily, weekly or monthly, or may be administered annually or even for longer periods of time. Suitable daily dosage regimen may include single or multiple servings per day.

Pharmaceutical, food or beverage incorporating fiber blends according to embodiments of the present disclosure can be safely-consumed. The fiber blends and nutritional compositions in embodiments of the present disclosure may be particularly suitable for the following:

Control of inflammatory bowel disease

Control of irritable bowel syndrome

Chemo induced diarrhea

Radio therapy induced diarrhea

Chemo-radio combination induced diarrhea

Reduction in eczema and other atopic diseases

Modulation of IgE

Maintenance of SIgA for reduction of infections

Reduction in allergies and allergic reaction

Reduction in asthma

### EXAMPLES

By way of example and not limitation, the following examples are illustrative of various embodiments of the present disclosure.

### Example 1

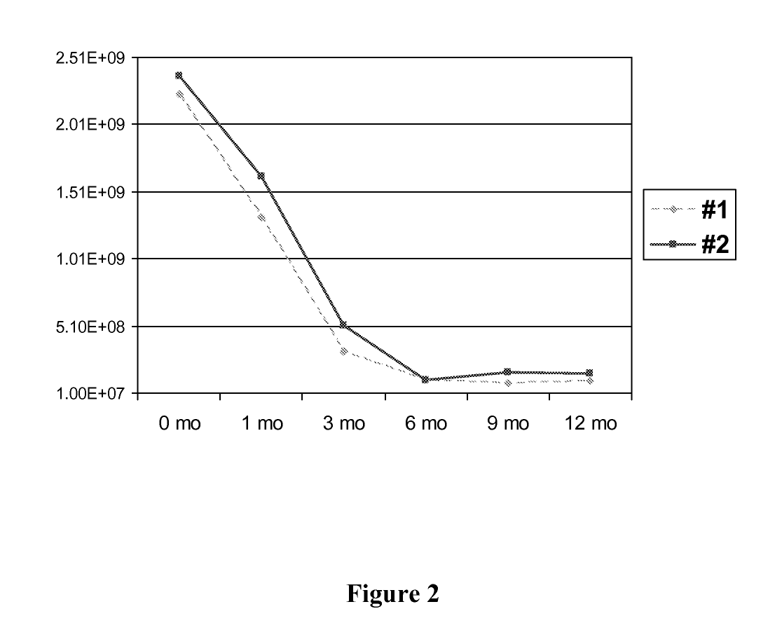

The viability of a probiotic in a typical fiber blend (e.g. Benefiber® HP Gel and Fibruline® XL) having powdered fiber (see Table 1) and the probiotic (L. reuteri) was compared to the viability of the probiotic in the improved fiber blend having agglomerated fiber particulates and the probiotic (see Table 2). The results are shown in FIG. 2. As seen in FIG. 2 and the tables, the data shows that the water activity for the improved fiber blend is maintained at a lower level, which allows for the better maintenance of viability of the probiotic component over time. The amount of the probiotic is 1×10⁸ CFU/sachet, which is the defined effective dose for L. reuteri.

TABLE 1
Fiber blend #1 including powder fiber and probiotic
30° C.

| | Month | | | | | |
|---|---|---|---|---|---|---|
| | 0 | 1 | 3 | 6 | 9 | 12 |
| CFU/g | 4.45E+08 | 2.63E+08 | 6.42E+07 | 2.10E+07 | 1.72E+07 | 1.96E+07 |
| CFU/sachet | 2.23E+09 | 1.32E+09 | 3.21E+08 | 1.05E+08 | 8.60E+07 | 9.80E+07 |
| Water | 0.13 | 0.13 | 0.13 | 0.14 | 0.18 | 0.17 |
| acitivity | | | | | | |

TABLE 1
Fiber blend #2 including agglomerated fiber particulates and probiotic
30° C.

| | Month | | | | | |
|---|---|---|---|---|---|---|
| | 0 | 1 | 3 | 6 | 9 | 12 |
| CFU/g | 4.74E+08 | 3.24E+08 | 1.03E+08 | 2.13E+07 | 3.23E+07 | 3.05E+07 |
| CFU/sachet | 2.37E+09 | 1.62E+09 | 5.15E+08 | 1.07E+08 | 1.62E+08 | 1.53E+08 |
| Water | 0.10 | 0.10 | 0.09 | 0.11 | 0.12 | 0.11 |
| acitivity | | | | | | |

An embodiment of the present invention is intended to include a complete nutrition product. As used herein, “complete nutrition” are preferably nutritional products that contain sufficient types and levels of macronutrients (protein, fats and carbohydrates) and micronutrients to be sufficient to be a sole source of nutrition for the animal to which it is being administered to. Patients can receive 100% of their nutritional requirements from such complete nutritional compositions.

An embodiment of the present invention is intended to include an incomplete nutrition product. As used herein, “incomplete nutrition” are preferably nutritional products that do not contain sufficient levels of macronutrients (protein, fats and carbohydrates) or micronutrients to be sufficient to be a sole source of nutrition for the animal to which it is being administered to. Partial or incomplete nutritional compositions can be used as a nutritional supplement.

An embodiment of the present invention is intended for short term administration. As used herein, “Short term administrations” are preferably continuous administrations for less than 6 weeks.

An embodiment of the present invention is intended for long term administration. As used herein, “Long term administrations” are preferably continuous administrations for more than 6 weeks.

As used in this specification and the appended claims, the singular forms “a”, “an” and “the” include plural referents unless the context clearly dictates otherwise. Thus, for example, reference to “a polypeptide” includes a mixture of two or more polypeptides, and the like.

As used herein, “about,” is preferably understood to refer to numbers in a range of numerals. Moreover, all numerical ranges herein should be understood to include all integer, whole or fractions, within the range.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A nutritional composition comprising:
a fiber blend comprising agglomerated fiber particulates and a probiotic, the fiber blend having a water activity of less than about 0.2.

2. The nutritional composition of claim 1, wherein the water activity of the fiber blend is less than about 0.15.

3. The nutritional composition of claim 1, wherein the agglomerated fiber particulates comprise a fiber selected from the group consisting of fructooligosaccharides, inulin, galactooligosaccharides, partially hydrolyzed guar gum, galactomannans, acacia gum, pectins, arabinogalactans, beta-glucans, xanthan gum and combinations thereof.

4. The nutritional composition of claim 1, wherein the agglomerated fiber particulates comprise an average diameter ranging between about 1.2 mm to about 5 mm.

5. The nutritional composition of claim 1, wherein the water activity of the fiber blend is less than about 0.12.

6. The nutritional composition of claim 1, wherein the probiotic is selected from the group consisting of Aerococcus, Aspergillus, Bacillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, or a combination thereof.

7. The nutritional composition of claim 1, wherein the probiotic is selected from the group consisting of Lactobacillus reuteri, Lactobacillus rhamnoses, Lactobacillus plantarum and combinations thereof.

8. The nutritional composition of claim 1, wherein the probiotic is Lactobacillus reuteri.

9. The nutritional composition of claim 1, wherein the fiber blend further comprises a metabolitegenerated by the probiotic during a fermentation process.

10. The nutritional composition of claim 1 further comprising an ingredient selected from the group consisting of vitamins, minerals, proteins, bioactives, phytonutrients, antioxidants and combinations thereof.

11. The nutrition composition of claim 1, wherein the nutritional composition is in an administerable form selected from the group consisting of pharmaceutical formulations, nutritional formulations, dietary supplements, functional foods and beverage products.

12. A method for making a nutrition composition, the method comprising:
providing a fiber;
agglomerating the fiber to produce agglomerated fiber particulates having a water activity of less that 0.2; and
mixing a probiotic with the agglomerated fiber particulates to produce a fiber blend.

13. The method of claim 12 further comprising storing the fiber blend in a nitrogen atmosphere.

14. The method of claim 12, wherein the fiber is premixed with a powdered form of the fiber before being mixed with the agglomerated fiber particulates.

15. The method of claim 12, wherein the agglomerated fiber particulates comprise a fiber selected from the group consisting of fructooligosaccharides, inulin, galactooligosaccharides, partially hydrolyzed guar gum, galactomannans, acacia gum, pectins, arabinogalactans, beta-glucans, xanthan gum and combinations thereof.

16. The method of claim 12, wherein the agglomerated fiber particulates comprise an average diameter ranging between about 1.2 mm to about 5 mm.

17. The method of claim 12, wherein the water activity of the fiber blend is less than about 0.15.

18. The method of claim 12, wherein the water activity of the fiber blend is less than about 0.12.

19. The method of claim 12, wherein the probiotic is selected from the group consisting of Aerococcus, Aspergillus, Bacillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, or a combination thereof.

20. The method of claim 12, wherein the probiotic is selected from the group consisting of Lactobacillus reuteri, Lactobacillus rhamnoses, Lactobacillus plantarum and combinations thereof.

21. The method of claim 12, wherein the probiotic is Lactobacillus reuteri.

22. The method of claim 12, wherein the fiber blend further comprises a metabolite generated by the probiotic during a fermentation process.

23. The method of claim 12 further comprising an ingredient selected from the group consisting of vitamins, minerals, proteins, bioactives, phytonutrients, antioxidants and combinations thereof.
